(19) United States
(12) Patent Application Publication
(10) Pub. No.: US 2018/0304053 A1
(43) Pub. Date: Oct. 25, 2018

(54) Lithotripsy Angioplasty Devices and Methods

(71) Applicant: BOSTON SCIENTIFIC SCIMED, INC., Maple Grove, MN (US)

(72) Inventors: Joel T. Eggert, Plymouth, MN (US); Douglas Dean Pagoria, Forest Lake, MN (US); Raymond Gessler, Roberts, WI (US); Douglas Pennington, Stillwater, MN (US); Daniel J. Foster, Lino Lakes, MN (US); James P. Rohl, Prescott, WI (US)

(73) Assignee: BOSTON SCIENTIFIC SCIMED, INC., Maple Grove, MN (US)

(21) Appl. No.: 15/950,717

(22) Filed: Apr. 11, 2018

Related U.S. Application Data
(60) Provisional application No. 62/488,409, filed on Apr. 21, 2017.

(51) Int. Cl. A61M 25/10; A61B 18/14

(52) U.S. Cl. CPC A61M 25/104; A61B 2017/22001; A61B 18/1492

(57) ABSTRACT

Medical devices and method for making and using medical devices are disclosed. An example method for treating a blood vessel may include disposing a medical device within the blood vessel at a position adjacent to a lesion. The medical device may comprise an elongate shaft having a distal end region, a balloon coupled to the distal end region, and a force transmitting member at least partially disposed at least partially within the balloon. The force transmitting member may be designed to transmit energy to the lesion. The method may also include inflating the balloon to a first pressure, actuating the force transmitting member to at least partial break apart the lesion, and inflating the balloon to a second pressure greater than the first pressure.

### CROSS REFERENCE TO RELATED APPLICATIONS

This application claims the benefit of U.S. Provisional Patent Application Ser. No. 62/488,409 filed on Apr. 21, 2017, the disclosure of which is incorporated herein by reference.

### TECHNICAL FIELD

The present disclosure pertains to medical devices, and methods for manufacturing medical devices. More particularly, the present disclosure pertains to lithotripsy angioplasty devices and methods.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example method for treating a blood vessel is disclosed. The method comprises: disposing a medical device within the blood vessel at a position adjacent to a lesion, the medical device comprising: an elongate shaft having a distal end region, a balloon coupled to the distal end region, and a force transmitting member at least partially disposed within the balloon, the force transmitting member being designed to transmit energy to the lesion; inflating the balloon to a first pressure; actuating the force transmitting member to at least partial break apart the lesion; and inflating the balloon to a second pressure greater than the first pressure.

Alternatively or additionally to any of the embodiments above, the force transmitting member includes one or more electrode, and wherein actuating the force transmitting member to at least partial break apart the lesion includes activating the one or more electrode.

Alternatively or additionally to any of the embodiments above, the one or more electrode includes a bipolar electrode pair and wherein activating the one or more electrode includes activating the bipolar electrode pair.

Alternatively or additionally to any of the embodiments above, the one or more electrode are radiofrequency electrodes and wherein activating the one or more electrode includes transmitting radiofrequency energy at a frequency of 3-30 hertz.

Alternatively or additionally to any of the embodiments above, the one or more electrode includes a radiofrequency electrode and wherein activating the one or more electrode includes transmitting radiofrequency energy at a frequency of 300 gigahertz to 3 terahertz.

Alternatively or additionally to any of the embodiments above, the force transmitting member includes a plurality of fluid jets positioned under the balloon and in fluid communication with an external pump, and wherein actuating the force transmitting member to at least partial break apart the lesion includes rapidly inflating and deflating the balloon by cycling the pump.

Alternatively or additionally to any of the embodiments above, the force transmitting member includes an ultrasound transducer positioned under the balloon, and wherein actuating the force transmitting member to at least partial break apart the lesion includes activating the ultrasound transducer.

Alternatively or additionally to any of the embodiments above, the force transmitting member includes an external ultrasound generator in fluid communication with the balloon, and wherein actuating the force transmitting member to at least partial break apart the lesion includes activating the ultrasound generator to generate a fluid pulse within the balloon.

A method for treating a blood vessel is disclosed. The method comprises: disposing a lithotripsy angioplasty medical device within the blood vessel at a position adjacent to a calcified lesion, the lithotripsy angioplasty medical device comprising: an elongate shaft having a distal end region, a balloon coupled to the distal end region, and a force transmitting member at least partially disposed at least partially within the balloon, the force transmitting member being designed to transmit energy to the calcified lesion; inflating the balloon to a first pressure; transferring force from the balloon to the calcified lesion by activing the force transmitting member in order to at least partial break apart the calcified lesion; and inflating the balloon to a second pressure greater than the first pressure.

Alternatively or additionally to any of the embodiments above, the force transmitting member includes one or more electrode, and wherein transferring force from the balloon to the calcified lesion includes activating the one or more electrode.

Alternatively or additionally to any of the embodiments above, the one or more electrode includes a bipolar electrode pair.

Alternatively or additionally to any of the embodiments above, the force transmitting member includes a plurality of fluid jets positioned under the balloon and in fluid communication with an external pump, and wherein transferring force from the balloon to the calcified lesion includes rapidly inflating and deflating the balloon by cycling the pump.

Alternatively or additionally to any of the embodiments above, the force transmitting member includes an ultrasound transducer, and wherein transferring force from the balloon to the calcified lesion includes activating the ultrasound transducer.

A lithotripsy angioplasty medical device is disclosed. The lithotripsy angioplasty medical device comprises: an elongate shaft having a distal end region; a balloon coupled to the distal end region; one or more electrode coupled to the shaft and positioned under the balloon, the one or more electrode being designed to generate localized gas bubbles within the balloon in order to transmit energy to a target region; wherein the balloon is designed to shift between a first unexpanded configuration, a second configuration when the balloon is partially expanded into contact with the target region, and an expanded configuration.

Alternatively or additionally to any of the embodiments above, the elongate shaft includes an inner shaft and an outer shaft, wherein a proximal waist of the balloon is attached to the outer shaft, and wherein a distal waist of the balloon is attached to the inner shaft.

Alternatively or additionally to any of the embodiments above, the one or more electrode includes a single radiofrequency electrode.

Alternatively or additionally to any of the embodiments above, the one or more electrode includes a bipolar electrode pair.

Alternatively or additionally to any of the embodiments above, the one or more electrode includes a plurality of bipolar electrode pairs.

Alternatively or additionally to any of the embodiments above, the one or more electrode being designed to transmit radiofrequency energy at a frequency of 3-30 hertz.

Alternatively or additionally to any of the embodiments above, the one or more electrode being designed to transmit radiofrequency energy at a frequency of 300 gigahertz to 3 terahertz.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional side view of an example medical device.

FIG. 2 is a cross-sectional side view of a portion of a blood vessel.

FIG. 3 is a partial cross-sectional side view of an example medical device disposed in a blood vessel.

FIG. 4 is a partial cross-sectional side view of an example medical device disposed in a blood vessel.

FIG. 5 is a partial cross-sectional side view of an example medical device disposed in a blood vessel.

FIG. 6 is a partial cross-sectional side view of an example medical device disposed in a blood vessel.

FIG. 7 is a partial cross-sectional side view of an example medical device disposed in a blood vessel.

FIG. 8 is a partial cross-sectional side view of an example medical device disposed in a blood vessel.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

The use of medical devices for balloon angioplasty may be a desirable method for treating intravascular lesions in blood vessel. In some instances, calcification along or otherwise adjacent to the vessel wall can complicate an intervention. Disclosed herein are angioplasty devices and methods that are designed to improve the treatment of intravascular lesions. The devices and methods disclosed herein may be described as lithotripsy angioplasty devices/methods in that the devices may transfer a force to the treatment area to break up the lesion. Some additional details are disclosed herein.

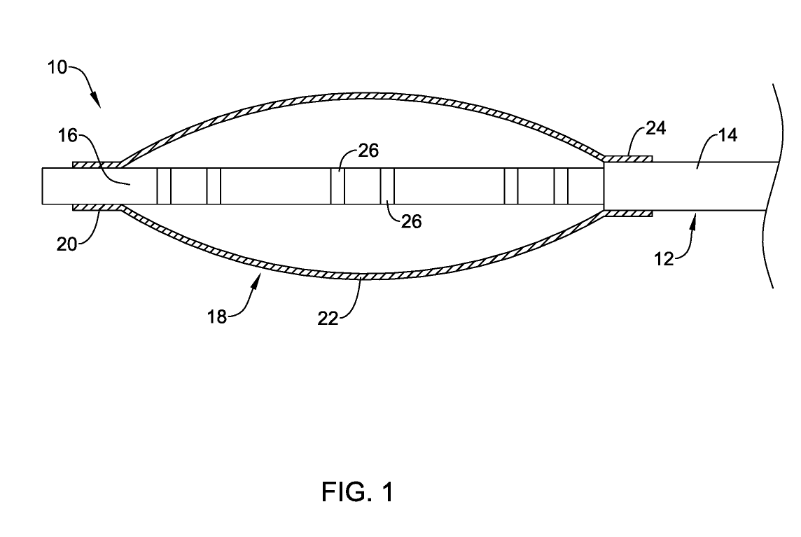

FIG. 1 is a partial cross-sectional side view of an example medical device 10. The medical device 10 may include a catheter shaft 12. In some instances, the catheter shaft 12 may include a first or outer member 14 and a second or inner member 16. A balloon 18 may be coupled to the catheter shaft 12. In some instances, the balloon 18 may include a distal waist 20, a body region 22, and a proximal waist 24. The distal waist 20 may be coupled to the inner member 16. The proximal waist 24 may be coupled to the outer member 14. Other constructions are contemplated.

In at least some instances, the medical device 10 may be considered a lithotripsy medical device and/or a lithotripsy angioplasty medical device. For the purposes of this disclosure, a lithotripsy angioplasty device may be understood to a device designed to transfer forces to a target region in a manner that may break up the target region. In at least some instances, the transfer of forces may occur in a repeated manner with waves or flurries of force that are meant to impact the target region. The repeated force transfers could occur in a regular manner with equally spaced time intervals between transfers, or the force transfers could occur with differing time intervals between transfers. While lithotripsy devices may be commonly associated with ultrasound and/or ultrasonic waves, the lithotripsy angioplasty devices disclosed herein are not meant to be limited to ultrasound devices. Indeed, medical device 10 (as well as other medical devices disclosed herein) may use ultrasound and/or other force generators to transfer force to the target site.

The medical device 10 may include one or more force transferring member(s) 26. In this example, the force transferring members 26 may include one or more electrode(s) 26 coupled to the shaft 12 (e.g., the inner member 16). A lead (not shown) may be coupled to the electrodes 26 and extend to a power supply or generator. In some instances, the electrodes 26 may take the form of monopolar electrodes. Alternatively, the electrodes 26 may form one or more pairs of bipolar electrodes (and/or form bipolar electrode pairs). In general, the number, spacing, arrangement, and configuration of the electrodes 26 can vary. For example, the medical device 10 may include one, two, three, four, five, six, seven, eight or more electrodes 26. Some or all of the electrodes may be arranged as bipolar pairs. The spacing between the electrodes may be regular/even or uneven. The electrodes 26 may be used with radiofrequency (RF) energy or another suitable energy. Activating the electrodes 26, for example with pulses of RF energy, may create localized gas bubbles within the balloon 18, which may cause the balloon 18 to acutely increase its volume and expand. Because the balloon 18 may be under relatively high pressure, the bubble may go rapidly back into solution. The expansion of the balloon 18 can transmit mechanical energy and/or a mechanical force to a target region. For example, RF energy may be pulse to cause repeated pulses of force that can be transferred to the target region. In cases where the target region is a calcified lesion, the force transfer can break up the calcified lesion.

In some instances, the frequency of RF energy used to activate the electrodes 26 may vary. For example, in some instances, frequencies from the tremendously high frequency (THF) band may be used (e.g., on the order of about 300 gigahertz to 3 terahertz). Such frequencies may be suitable when higher energy is needed to break up a lesion. In other instances, frequencies from the extremely low frequency (ELF) band may be used (e.g., on the order of about 3-30 hertz). Such frequencies may be utilized when a lower energy is suitable to break up a lesion. Other frequencies are contemplated including frequencies overlapping with and between the THF and ELF bands.

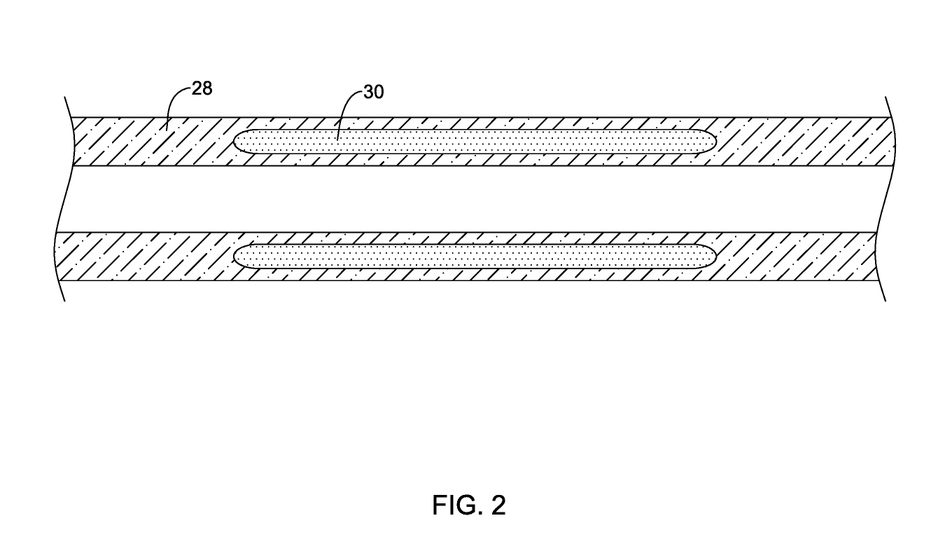

FIGS. 2-5 illustrate the use of the medical device 10. For example, FIG. 2 illustrates an example blood vessel 28. A calcified lesion 30 may be disposed along the blood vessel 28. In this example, the calcified lesion 30 is shown within the wall of the blood vessel 28. However, other arrangements may be seen. For example, portions or all of the calcified lesion 30 may be disposed along an inner surface of the blood vessel 28. In some of these and in other instances, plaque, a stenosis, a fatty deposit, or other types of lesions may also be present within the blood vessel 28.

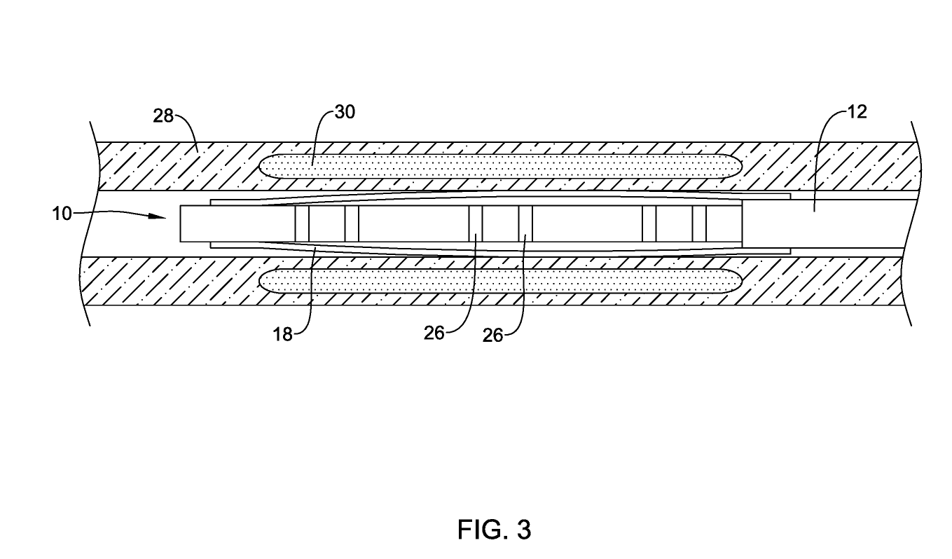

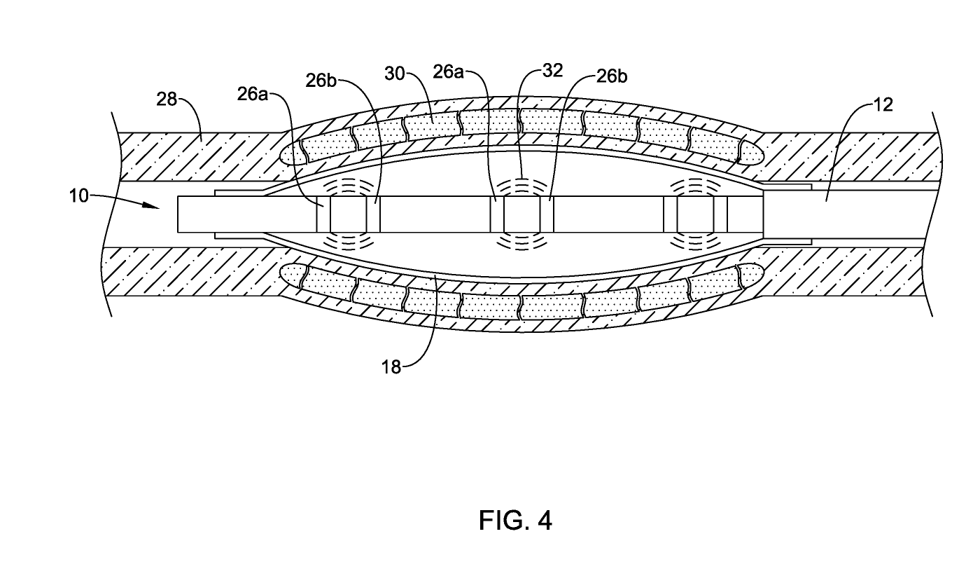

The medical device 10 may be advanced through the blood vessel 28 to a position adjacent to the calcified lesion 30 as shown in FIG. 3. When suitably positioned, the balloon 18 may be partially inflated as schematically depicted in FIG. 4. Partially inflating the balloon 18 may occur by infusing an inflation media into the balloon 18 (e.g., via an inflation lumen) that may be defined between the outer member 14 and the inner member 16. Partially inflating the balloon 18 may include inflating the balloon 18 so that the balloon 18 comes into contact with the wall of the blood vessel 28. This may include simply contacting the vessel wall or, in some instances, partially inflating the balloon 18 may include partially expanding the blood vessel 28. In some instances, partially inflating the balloon 18 may include inflating the balloon 18 to a first pressure that might be in the range of about 1-6 atmospheres or about 3-5 atmospheres.

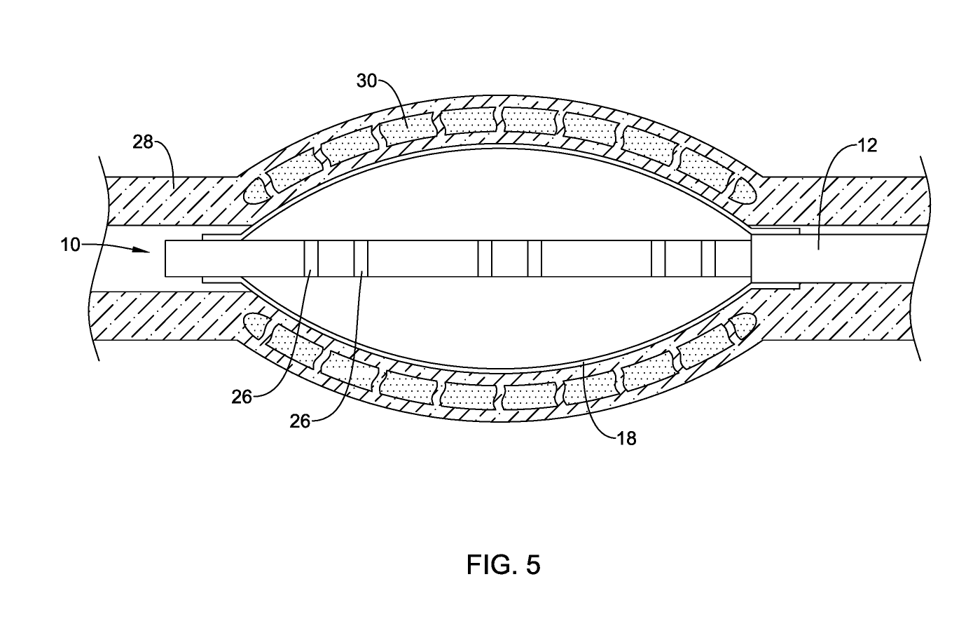

With the balloon 18 partially inflated, the force transferring members 26 may be activated. In FIG. 4, the electrodes 26 are labeled as bipolar electrode pairs 26a/26b. Activating the electrode pairs 26a/26b, including pulsing RF energy, may create a force or pulses of force 32 that can be transferred to the calcified lesion 30. The transferred forces may contact and break up the calcified lesion 30. With the calcified lesion 30 broken up, the balloon 18 may be further inflated to a second pressure, greater than the first pressure, to treat the blood vessel 28 as shown in FIG. 5. In some instances, the second pressure may be on the order of about 4-12 atmospheres or about 5-9 atmospheres.

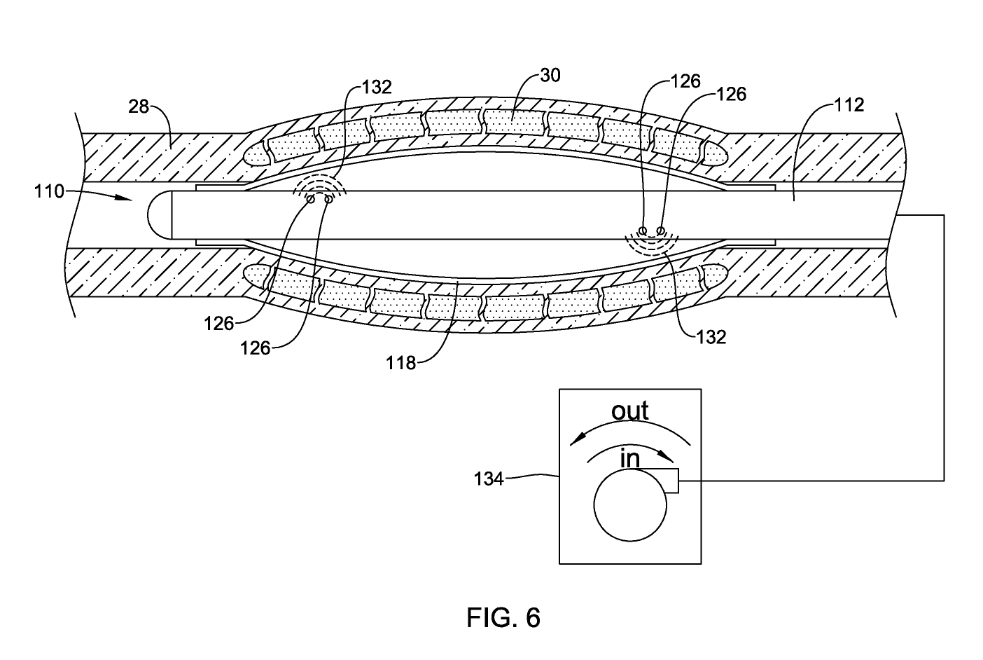

FIG. 6 illustrates another example medical device 110 disposed in the blood vessel 28. The medical device 110 may be similar in form and function to other medical devices disclosed herein. For example, the medical device 110 may include a catheter shaft 112, a balloon 118, and a force transmitting member 126. In this example, the force transmitting member 126 takes the form of a plurality of openings 126 in the catheter shaft 112 that allow fluid to be pumped into and out from the balloon 118. For example, a pump 134, schematically shown in FIG. 6, may be coupled to the catheter shaft 112 for hydraulically pulsing fluid. The pump 134 may be designed to rapidly cycle fluid in and out of the balloon 118 to generate a force 132. For example, the pump 134 may be programmed to pump fluid into and out of the balloon 118, the pump 134 may be manually switched in order to pump fluid into and out of the balloon 118, etc. The medical device 110 may be used similarly to other medical devices disclosed herein. For example, the medical device 110 may be advanced within the blood vessel 28 to a position adjacent to the calcified lesion 30, the balloon 118 may be partially inflated, the pump 134 may be used to cycle fluid into and out from the balloon 118 (e.g., via the openings 126) to create the force 132, and the balloon 118 may be further inflated (e.g., when the calcified lesion 30 is sufficiently broken up).

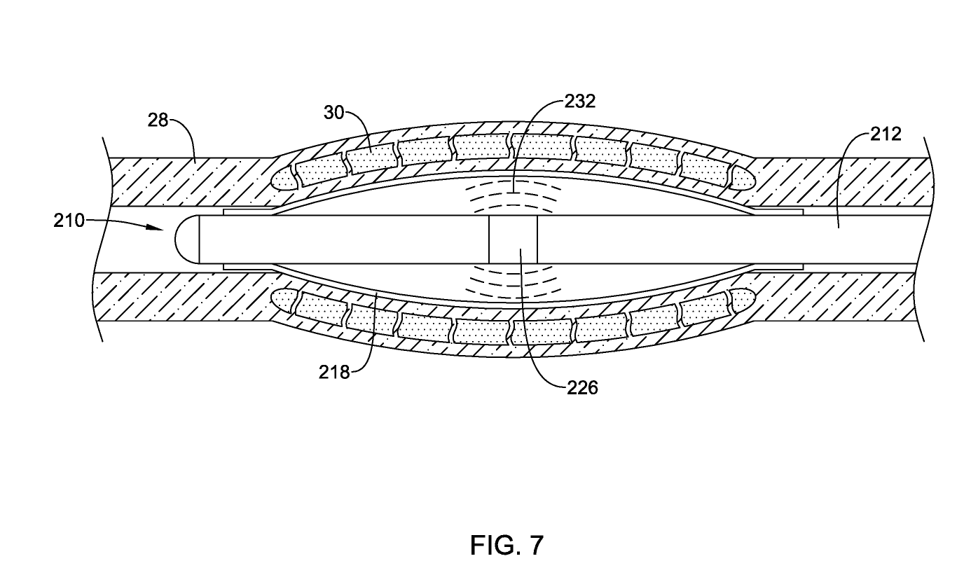

FIG. 7 illustrates another example medical device 210 disposed in the blood vessel 28. The medical device 210 may be similar in form and function to other medical devices disclosed herein. For example, the medical device 210 may include a catheter shaft 212, a balloon 218, and a force transmitting member 226. In this example, the force transmitting member 226 takes the form of an ultrasound transducer 226 (e.g., a piezoelectric ultrasound transducer) disposed within the balloon 218. In some instances, the ultrasound transducer 226 is coupled to the shaft 212. A lead (not shown) may be coupled to the ultrasound transducer 226 and extend to a power supply or generator. The force transmitting member 226 may be designed to transfer a force 232 onto the calcified lesion 30 to break up the calcified lesion 30. For example, the ultrasound transducer 226 may generate ultrasonic waves of energy that can cause cavitation within the balloon 218, which may transmit kinetic energy to the calcified lesion 30. The medical device 210 may be used similarly to other medical devices disclosed herein. For example, the medical device 210 may be advanced within the blood vessel 28 to a position adjacent to the calcified lesion 30, the balloon 218 may be partially inflated, the ultrasound transducer 226 may be activated to create the force 232, and the balloon 218 may be further inflated (e.g., when the calcified lesion 30 is sufficiently broken up).

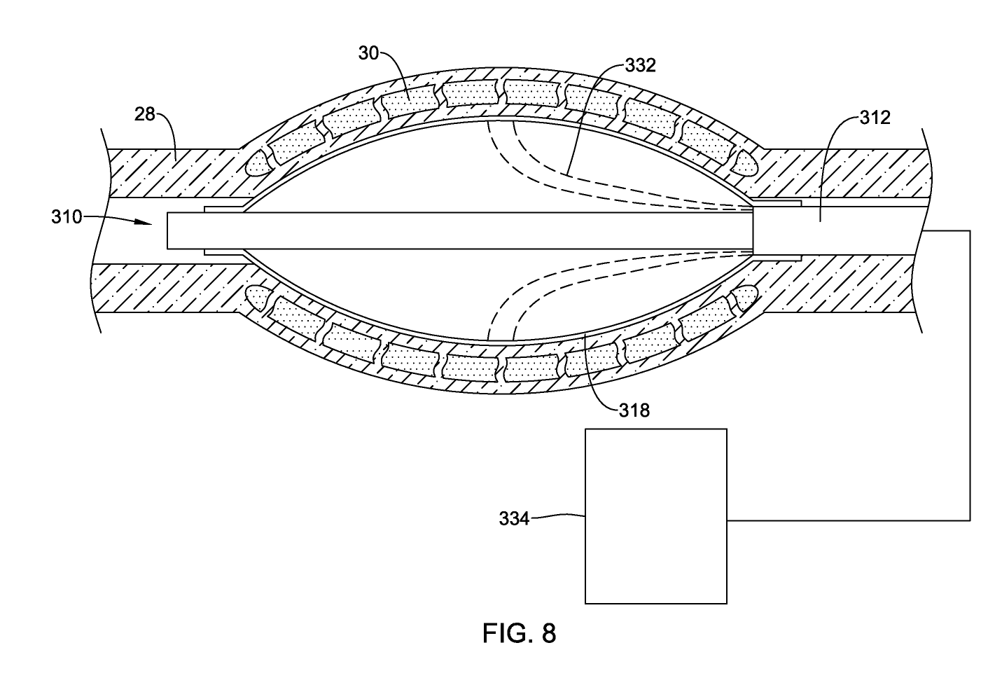

FIG. 8 illustrates another example medical device 310 disposed in the blood vessel 28. The medical device 310 may be similar in form and function to other medical devices disclosed herein. For example, the medical device 310 may include a catheter shaft 312 and a balloon 318. An external generator 334, schematically depicted in FIG. 8, may be coupled to the catheter shaft 312. The generator 334 may be an ultrasound generator. The generator 334 may be designed to generate a force 332. The medical device 310 may be used similarly to other medical devices disclosed herein. For example, the medical device 310 may be advanced within the blood vessel 28 to a position adjacent to the calcified lesion 30, the balloon 318 may be partially inflated, the generator 334 may be used to propagate ultrasonic energy into the balloon 118 to create the force 332, and the balloon 318 may be further inflated (e.g., when the calcified lesion 30 is sufficiently broken up).

The materials that can be used for the various components of the medical device 10, 110, 210, 310 (and/or other medical devices disclosed herein) disclosed herein may include those commonly associated with medical devices. For simplicity purposes, the following discussion makes reference to the catheter shaft 12, 112, 212, 312 and other components of applicable the medical device 10, 110, 210, 310. However, this is not intended to limit the devices and methods described herein, as the discussion may be applied to other similar tubular members and/or components of tubular members or devices disclosed herein.

The catheter shaft 12, 112, 212, 312 and/or other components of the medical device 10, 110, 210, 310 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device 10,110, 210, 310 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 10, 110, 210, 310 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 10, 110, 210, 310 to achieve the same result.

In some embodiments, a degree of Magnetic Resonance Imaging (MRI) compatibility is imparted into the medical device 10, 110, 210, 310. For example, the medical device 10, 110, 210, 310, or portions thereof, may be made of a material that does not substantially distort the image and create substantial artifacts (e.g., gaps in the image). Certain ferromagnetic materials, for example, may not be suitable because they may create artifacts in an MRI image. The medical device 10, 110, 210, 310, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method for treating a blood vessel, the method comprising:
disposing a medical device within the blood vessel at a position adjacent to a lesion, the medical device comprising:
an elongate shaft having a distal end region,
a balloon coupled to the distal end region, and
a force transmitting member at least partially disposed within the balloon, the force transmitting member being designed to transmit energy to the lesion;
inflating the balloon to a first pressure;
actuating the force transmitting member to at least partial break apart the lesion; and
inflating the balloon to a second pressure greater than the first pressure.

2. The method of claim 1, wherein the force transmitting member includes one or more electrode, and wherein actuating the force transmitting member to at least partial break apart the lesion includes activating the one or more electrode.

3. The method of claim 2, wherein the one or more electrode includes a bipolar electrode pair and wherein activating the one or more electrode includes activating the bipolar electrode pair.

4. The method of claim 2, wherein the one or more electrode includes radiofrequency electrodes and wherein activating the one or more electrode includes transmitting radiofrequency energy at a frequency of 3-30 hertz.

5. The method of claim 2, wherein the one or more electrode includes a radiofrequency electrode and wherein activating the one or more electrode includes transmitting radiofrequency energy at a frequency of 300 gigahertz to 3 terahertz.

6. The method of claim 1, wherein the force transmitting member includes a plurality of fluid jets positioned under the balloon and in fluid communication with an external pump, and wherein actuating the force transmitting member to at least partial break apart the lesion includes rapidly inflating and deflating the balloon by cycling the pump.

7. The method of claim 1, wherein the force transmitting member includes an ultrasound transducer positioned under the balloon, and wherein actuating the force transmitting member to at least partial break apart the lesion includes activating the ultrasound transducer.

8. The method of claim 1, wherein the force transmitting member includes an external ultrasound generator in fluid communication with the balloon, and wherein actuating the force transmitting member to at least partial break apart the lesion includes activating the ultrasound generator to generate a fluid pulse within the balloon.

9. A method for treating a blood vessel, the method comprising:
disposing a lithotripsy angioplasty medical device within the blood vessel at a position adjacent to a calcified lesion, the lithotripsy angioplasty medical device comprising:
an elongate shaft having a distal end region,
a balloon coupled to the distal end region, and
a force transmitting member at least partially disposed at least partially within the balloon, the force transmitting member being designed to transmit energy to the calcified lesion;
inflating the balloon to a first pressure;
transferring force from the balloon to the calcified lesion by activing the force transmitting member in order to at least partial break apart the calcified lesion; and
inflating the balloon to a second pressure greater than the first pressure.

10. The method of claim 9, wherein the force transmitting member includes one or more electrode, and wherein transferring force from the balloon to the calcified lesion includes activating the one or more electrode.

11. The method of claim 10, wherein the one or more electrode includes a bipolar electrode pair.

12. The method of claim 9, wherein the force transmitting member includes a plurality of fluid jets positioned under the balloon and in fluid communication with an external pump, and wherein transferring force from the balloon to the calcified lesion includes rapidly inflating and deflating the balloon by cycling the pump.

13. The method of claim 9, wherein the force transmitting member includes an ultrasound transducer, and wherein transferring force from the balloon to the calcified lesion includes activating the ultrasound transducer.

14. A lithotripsy angioplasty medical device, comprising:
an elongate shaft having a distal end region;
a balloon coupled to the distal end region;
one or more electrode coupled to the shaft and positioned under the balloon, the one or more electrode being designed to generate localized gas bubbles within the balloon in order to transmit energy to a target region;
wherein the balloon is designed to shift between a first unexpanded configuration, a second configuration when the balloon is partially expanded into contact with the target region, and an expanded configuration.

15. The lithotripsy angioplasty medical device of claim 14, wherein the elongate shaft includes an inner shaft and an outer shaft, wherein a proximal waist of the balloon is attached to the outer shaft, and wherein a distal waist of the balloon is attached to the inner shaft.

16. The lithotripsy angioplasty medical device of claim 14, wherein the one or more electrode includes a single radiofrequency electrode.

17. The lithotripsy angioplasty medical device of claim 14, wherein the one or more electrode includes a bipolar electrode pair.

18. The lithotripsy angioplasty medical device of claim 14, wherein the one or more electrode includes a plurality of bipolar electrode pairs.

19. The lithotripsy angioplasty medical device of claim 14, wherein the one or more electrode being designed to transmit radiofrequency energy at a frequency of 3-30 hertz.

20. The lithotripsy angioplasty medical device of claim 14, wherein the one or more electrode being designed to transmit radiofrequency energy at a frequency of 300 gigahertz to 3 terahertz.
